(19) United States
(12) Patent Application Publication
(10) Pub. No.: US 2009/0012274 A1
(43) Pub. Date: Jan. 8, 2009

(54) Unsaturated aldehyde surfaces and methods of molecular immobilization

(76) Inventor: Stephane Biltresse, Etterbeek (BE)

Correspondence Address: CORNING INCORPORATED, SP-TI-3-1, CORNING, NY, 14831, US

(21) Appl. No.: 12/080,908

(22) Filed: Apr. 7, 2008

Related U.S. Application Data
(60) Provisional application No. 60/958,502, filed on Jul. 6, 2007.

(51) Int. Cl. C07K 17/02; B32B 9/00; B32B 17/00; C09K 3/00

(52) U.S. Cl. 530/402; 428/704; 428/426; 428/336; 252/182.3

(57) ABSTRACT

The disclosure provides an article including a substrate having a surface modified with an unsaturated aldehyde, for example, of the formula:

≡Si—(Ar′)_{w}—(CH₂)ₓ—(CR═CR)_{y}—C(═O)H

where Ar′, R, w, x, and y are as defined herein. The disclosure also provides a method for making the article and a method of use of the article for immobilizing a biomolecule.

### CLAIMING BENEFIT OF PRIOR FILED U.S. APPLICATION

This application claims the benefit of U.S. Provisional Application Ser. No. 60/958,502, filed on Jul. 6, 2007. The content of this document and the entire disclosure of publications, patents, and patent documents mentioned herein are incorporated by reference.

### BACKGROUND

The disclosure relates to a method of making an unsaturated aldehyde on a substrate, such as an organic, an inorganic, or like surface, and more specifically to the use of such unsaturated aldehyde bearing substrate for biomolecular immobilization applications.

### SUMMARY

The disclosure provides a method of making an unsaturated aldehyde bearing substrate, and more specifically to the use of such unsaturated aldehyde substrates for biomolecular immobilization or like applications.

### BRIEF DESCRIPTION OF THE DRAWINGS

FIG. 1 shows exemplary and comparative protein immobilization results for modified surfaces, in embodiments of the disclosure.

### DETAILED DESCRIPTION

Various embodiments of the disclosure will be described in detail with reference to drawings, if any. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

### Definitions

“Attach,” “attachment,” “adhere,” “adhered,” “immobilized”, or like terms generally refer to immobilizing or fixing, for example, a protein or like synthetic or natural biological, a surface modifier substance, a compatibilizer, a cell, a ligand candidate compound, and like entities of the disclosure, to a surface, such as by physical absorption, chemical bonding, and like processes, or combinations thereof. In embodiments, covalent bonding of the unsaturated aldehyde to the substrate surface and covalent bonding of the immobilized protein on the unsaturated aldehyde modified substrate surface is preferred for stability and reproducibility considerations.

The indefinite article “a” or “an” and its corresponding definite article “the” as used herein means at least one, or one or more, unless specified otherwise.

“Include,” “includes,” or like terms means including but not limited to.

“About” modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to for example aging of a formulation with a particular initial concentration or mixture, and amounts that differ due to mixing or processing a formulation with a particular initial concentration or mixture. Whether modified by the term “about” the claims appended hereto include equivalents to these quantities.

“Consisting essentially of” in embodiments refers, for example, to a surface composition, a method of making or using a surface composition, formulation, or composition on the surface of a substrate or surface, such as a microplate or a biosensor, and articles, devices, or apparatus of the disclosure, and can include the components or steps listed in the claim, plus other components or steps that do not materially affect the basic and novel properties of the compositions, articles, apparatus, and methods of making and use of the disclosure, such as particular reactants, particular additives or ingredients, a particular agent, a particular surface modifier or condition, a particular ligand candidate, or like structure, material, or process variable selected. Items that may materially affect the basic properties of the components or steps of the disclosure or may impart undesirable characteristics to aspects of the present disclosure include, for example, decreased affinity of protein or like analyte molecules for the modified surface, decreased reactivity or the aldehyde modified surface for analyte molecules, and like characteristics.

Thus, the claimed invention may suitably comprise, consist of, or consist essentially of: a compound of the formula (I) or (II), or like compounds as defined herein; a composition of the reaction product including a compound of formula (I) or (II), or like compounds and a substrate, as defined herein; the reaction product including a modified substrate, as defined herein and a protein or like biomolecule, or a method of making or using the modified substrate as defined herein.

In embodiments, the disclosure provides a number of useful aspects. The protein immobilization efficiency on an unsaturated aldehyde modified surface of the disclosure is significantly improved compared to a saturated aldehyde modified surface, for example, from about 2 to about 10 fold greater protein immobilization can be achieved as measured by increased fluorescence. The unsaturated aldehyde modified surface and the immobilization method are versatile in that any suitable substrate (e.g., commercial, prepared, biological, or like material, and combinations thereof) having an aldehyde presenting group or aldehyde presenting surface may be used to form the corresponding unsaturated aldehyde modified surface of the disclosure. The process is relatively clean and straightforward to perform in an inert atmosphere, and can be accomplished without pressure or without a co-metallic reagent. Additionally, no reduction step is necessary after protein immobilization, so that lost time and additional potentially hazardous reagents are avoided. In embodiments, mild work-up conditions which avoid hydrolysis of the Schiff base are unnecessary.

In embodiments the disclosure provides a method of making an unsaturated aldehyde modified surface on a substrate from a substrate surface bearing a saturated aldehyde functional group. The substrate can be, for example, any suitable support material such as an organic material, inorganic material, or combinations thereof. The α,β-unsaturated aldehyde modified surfaces can be used for biomolecular immobilization applications where increased protein immobilization capacity is desired. The α,β-unsaturated aldehyde modified surfaces prepared in accordance with the disclosure provide higher immobilization capacities compared to their corresponding saturated aldehyde surface precursors or like surfaces. In embodiments protein immobilization accomplished with the α,β-unsaturated aldehyde modified surfaces of the disclosure permit one to forego the use of an optional reducing agent. Alternatively, use of an optional reducing agent can provide even greater increases in protein immobilization yields compared to immobilization yields obtained in the absence of a reducing agent.

In embodiments the disclosure provides an article comprising:

a substrate having a surface modified with an unsaturated aldehyde of the formula:

≡Si—(CH₂)ₓ—(CR═CR)_{y}—C(═O)H

where
the ≡Si valences are associated with the substrate,
R is independently H, a substituted or unsubstituted, saturated or unsaturated (C₁-C₆)alkyl, Ar, or Het,
x is from 1 to about 20, and
y is from 1 to about 3.

The substrate can be, for example, one of a glass, a plastic, a metal, a ceramer, a composite, and like materials, or combinations thereof. The surface modified with an unsaturated aldehyde on the substrate can have a thickness, for example, of from about 3 to about 100 nanometers, and from about 3 to about 50 nanometers. Other thicknesses are available and can depend upon the unsaturated aldehyde selected or created on the surface. In embodiments, the unsaturated aldehyde can be, for example, of the formula:

≡Si—(CH₂)ₓ—(CR═CR)_{y}—C(═O)H

where
the ≡Si valences are associated with the substrate,
R is independently H or saturated or unsaturated (C₁-C₆)alkyl,
x is from 1 to about 6, and
y is from 1 to about 3.

In embodiments, the unsaturated aldehyde can be, for example, of the formula:

≡Si—(CH₂)₃—CH═CH—C(═O)H

In embodiments the disclosure provides a composition comprising the reaction product of a compound of the formula (I) or formula (II):

(R¹O)₃Si—(Ar′)_{w}—(CH₂)ₓ—(CR═CR)_{y}—C(═O)H (I)

(R¹O)₃Si—(Ar′)_{w}—(CH₂)ₓ—(CR═CR)_{y}—R² (II)

where
R is independently H, a substituted or unsubstituted, saturated or unsaturated (C₁-C₆)alkyl, Ar, or Het,
R¹ is independently H or (C₁-C₆)alkyl,
R² is an acetal of the formula —C(—O—R)(—O—R)—H where the acetal R groups are independently (C₁-C₆)alkyl or a single conjoined cyclic R group such as —CH₂—CH₂— or —CH₂—CH₂—CH₂—,
Ar′ is aryl or Het,
w is from 1 to about 2,
x is from 1 to about 20, and
y is from 1 to about 3; and

a substrate comprised of at least one of a glass, a plastic, a metal, a ceramer, a composite, or combinations thereof.

In embodiments, R can be independently H, a substituted or unsubstituted, saturated or unsaturated (C₁-C₆)alkyl, R¹ can be (C₁-C₆)alkyl, R² can be an acetal of the formula —C(—O—R)(—O—R)—H where the acetal R groups are independently (C₁-C₆)alkyl or a single cyclic R group, w is can be from 0 to about 2,
x can be from 1 to about 10, and
y can be 1.

The compound of the formula (I) or formula (II) can be, for example, of the formula:

(R¹O)₃Si—(CH₂)₃—CH═CH—C(═O)H

or

(R¹O)₃Si—(CH₂)₃—CH═CH—C(—O—CH₂)₂H

In embodiments the disclosure provides a method of making an article, the method comprising:

reacting a substrate having a surface bearing a saturated aldehyde of the formula:

≡Si—(CH₂)ₓ—C(═O)H

where the —Si valences are associated with the substrate, and x is from 1 to about 10, with a “═CR—C(═O)—H” synthon to afford the article comprising a substrate having a surface modified with an α,β-unsaturated aldehyde of the formula

≡Si—(CH₂)ₓ—(CR═CR)_{y}—C(═O)H

where
R is independently H, a substituted or unsubstituted, saturated or unsaturated (C₁-C₆)alkyl, Ar, or Het,
x is from 1 to about 10, and
y is from 1 to about 3.

The “═CR—C(═O)—H” synthon can be, for example, an activated ylide precursor of at least one of:

a phosphonium salt of the formula

Ar₃P⁽⁺⁾—CR₂—R²X⁽⁻⁾

where
R is H, a substituted or unsubstituted, saturated or unsaturated (C₁-C₆)alkyl, Ar, or Het,
R² is an acetal of the formula —C(—O—R)(—O—R)—H where the acetal R groups can be, for example, independently (C₁-C₆)alkyl or a single conjoined cyclic R group, for example, a protected or masked aldehyde {—C(═O)H} such as masked group of the formula —C(—O—CH₂)₂H or —C(—O—CH₂CH₃)₂H,
Ar is aryl, and
X is a counter anion, such as a halide (Cl⁻, Br⁻, I⁻), hydroxide (⁻OH), or an alkoxide (RO⁻ where R is (C₁-C₆)alkyl), or like counterions;

a phosphonium salt of the formula

Y₃P⁽⁺⁾—CR₂—R²X⁽⁻⁾

where
R, R², and X are as defined above, and
each Y is independently a substituted or unsubstituted (C₁-C₆)alkyl, a substituted or unsubstituted oxygenated (C₁-C₆)alkyl, such as an ether substituent or alkylated glycol substituent (e.g., CH₃—CH₂—O—CH₂—CH₂—, CH₃—O—CH₂—CH₂—O—CH₂—, CH₃—O—CH₂—CH₂—O—, CH₃—O—CH—(CH₃)—CH₂—O—, (CH₃)₂CH—O—CH₂—CH₂—CH₂—O—, and like groups), a substituted or unsubstituted C₁₋₇alkoxy, a protected substituted or unsubstituted C₂₋₇alkanoyl, or where two Y groups form a cyclic (C₃-C₆)alkyl group or oxygenated cyclic (C₂-C₆)alkyl group (e.g., —CH₂—O—CH₂—CH₂—O—CH₂—, —O—CH—(CH₃)—CH₂—O—CH₂—, —O—CH—(CH₃)—CH₂—O—, and like groups);

a phosphonate of the formula

(RO)₂P(═O)—CR═CH—NH—R³

where
R is H, a substituted or unsubstituted, saturated or unsaturated (C₁-C₆)alkyl, Ar, or Het, and R³ is (C₁-C₆)alkyl, (C₁-C₆)cyloalkyl, or Ar, or combinations thereof.

The foregoing Wadsworth-Emmons modification of the Wittig reaction may conveniently avoid problems often encountered in separating the triphenylphosphine oxide byproduct from the olefin or the unsaturated carbonyl product in the usual Wittig procedure. See for example, W. Nagata and Y. Hayase, J. Chem. Soc., C, 460 (1969) where an enamime phosphonate was treated with a base (e.g., NaH, THF, 0° C.) to form the corresponding ylide. The ylide was reacted with a carbonyl compound (e.g., keto-steroid, THF, 25° C.) to form an imine phosphonate which was then hydrolyzed (e.g., oxalic acid, H₂O, benzene, reflux) to the α,β-unsaturated aldehyde.

An “activated” ylide precursor refers to ylide precursor that has been, for example, deprotonated with a suitable base to form the ylide and is in a condition to react with reactive surface carbonyl groups. The substrate has functional groups thereon that are reactive with compound of the formula (I) or formula (II), the functional groups can be, for example, hydroxyl, amine, hydrazide, and like groups, or mixtures thereof.

In embodiments the disclosure provides a method of making an article comprising:

reacting a substrate with a compound of the formula (I) or formula (II):

(R¹O)₃Si—(Ar′)_{w}—(CH₂)ₓ—(CR═CR)_{y}—C(═O)H (I)

(R¹O)₃Si—(Ar′)_{w}—(CH₂)ₓ—(CR═CR)_{y}—R² (II)

where
R is independently H, a substituted or unsubstituted, saturated or unsaturated (C₁-C₆)alkyl, Ar, or Het,
R¹ is independently H or (C₁-C₆)alkyl,
R² is an acetal of the formula —C(—O—R)(—O—R)—H where the acetal R groups are independently (C₁-C₆)alkyl or a conjoined cyclic R group,
x is from 1 to about 10, and
y is from 1 to about 3;
   the substrate comprises at least one of a glass, a plastic, a metal, a ceramer, a composite, or combinations thereof. In embodiments, the preparative method can include a step to remove the R² acetal protecting group if necessary. In embodiments the method of making the article can further comprise hydrolyzing the intermediate acetal product resulting from formula (II).

The substrate preferably has one or more functional groups thereon that are reactive with a compound of the formula (I) or formula (II), the functional groups can be selected from, for example, hydroxyl (—OH), amine (—NH₂ or —NHR), thiol (—SH), hydrazide (—R⁴R⁵N—NH₂ where at least one of R⁴ or R⁵ is acyl including carbonyl, sulfonyl and phosphonyl derivatives), hydrazine (—R⁴R⁵N—NH₂), and like groups, or combinations thereof.

In embodiments the disclosure provides an article having an unsaturated aldehyde on the surface of the article and a method for preparing the article. For example, a saturated aldehyde present on a substrate surface, such as an organic or inorganic material, can be reacted with an organophosphorous reagent to form an unsaturated aldehyde. The reaction conditions are mild and reproducible. The method can be used to modify any substrate having a saturated aldehyde surface into an unsaturated aldehyde surface.

In embodiments the disclosure provides a method of immobilizing biomolecules, the method comprising:

contacting the article having a substrate surface modified with an unsaturated aldehyde with a sample containing a biomolecule; and optionally

rinsing and drying the contacted article.

The biomolecule can be, for example, a protein, nucleic acid, an antibody, and like materials having at least one suitably reactive functional group such as an amine or and amino acid.

In embodiments, halo or halide includes fluoro, chloro, bromo, or iodo. Alkyl, alkoxy, etc., include both straight and branched groups; but reference to an individual radical such as “propyl” embraces only the straight chain radical, a branched chain isomer such as “isopropyl” being specifically referred to.

“Alkyl” includes linear alkyls, branched alkyls, and cycloalkyls.

“Substituted alkyl” or “optionally substituted alkyl” refers to an alkyl substituent, which includes linear alkyls, branched alkyls, or cycloalkyls, having from 1 to 4 optional substituents selected from, for example, hydroxyl (—OH), halogen, amino (—NH₂), nitro (—NO₂), alkyl, acyl (—C(═O)R), alkylsulfonyl (—S(═O)₂R) or alkoxy (—OR). For example, an alkoxy substituted alkyl, can be a 2-methoxy substituted ethyl of the formula —CH₂—CH₂—O—CH₃, a 1-dialkylamino substituted ethyl of the formula —CH₂(NR₂)—CH₃, and like substituted alkyl substituents.

“Aryl” includes a mono- or divalent-phenyl radical or an ortho-fused bicyclic carbocyclic radical having about nine to twenty ring atoms in which at least one ring is aromatic. Aryl (Ar) can include substituted aryls, such as a phenyl radical having from 1 to 5 substituents, for example, alkyl, alkoxy, halo, and like substituents.

“Het” includes a four-(4), five-(5), six-(6), or seven-(7) membered saturated or unsaturated heterocyclic ring having 1, 2, 3, or 4 heteroatoms selected from the group consisting of oxy, thio, sulfinyl, sulfonyl, and nitrogen, which ring is optionally fused to a benzene ring. Het also includes “heteroaryl,” which encompasses a radical attached via a ring carbon of a monocyclic aromatic ring containing five or six ring atoms consisting of carbon and 1, 2, 3, or 4 heteroatoms each selected from the group consisting of non-peroxide oxy, thio, and N(X) wherein X is absent or is H, O, (C₁₋₄)alkyl, phenyl, or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto.

The carbon atom content of various hydrocarbon-containing moieties is indicated by a prefix designating a lower and upper number of carbon atoms in the moiety, i.e., the prefix Cᵢ₋ⱼ indicates a moiety of the integer “i” to the integer “j” carbon atoms, inclusive. Thus, for example, (C₁-C₇)alkyl or C₁₋₇alkyl refers to alkyl of one to seven carbon atoms, inclusive, and (C₁-C₄)alkyl or C₁₋₄alkyl refers to alkyl of one to four carbon atoms, inclusive.

The compounds of the present disclosure are generally named according to the IUPAC nomenclature system. Abbreviations, which are well known to one of ordinary skill in the art, may be used (e.g., “Ph” for phenyl, “Me” for methyl, “Et” for ethyl, “h” or “hrs” for hour or hours, “g” or “gm” for gram(s), “mL” for milliliters, and “rt” for room temperature, “nm” for nanometers, and like abbreviations).

Specific and preferred values listed below for radicals, substituents, components, ingredients, additives, and like aspects, and ranges thereof, are for illustration only; they do not exclude other defined values or other values within defined ranges for the radicals and substituents. The compounds of the disclosure include compounds of formula (I) and like compounds having any combination of the values, specific values, more specific values, and preferred values described herein.

Specifically, (C₁₋₄)alkyl can be methyl, ethyl, propyl, isopropyl, butyl, iso-butyl, sec-butyl, tert-butyl; (C₁-C₆)alkyl can be methyl, ethyl, propyl, isopropyl, butyl, iso-butyl, sec-butyl, tert-butyl, pentyl, 3-pentyl, or hexyl; (C₃₋₁₂)cycloalkyl can be cyclopropyl, cyclobutyl, cyclopentyl, cyclohexyl, cycloheptyl, cyclooctyl, bicyclic, or multi-cyclic substituents, such as of the formulas

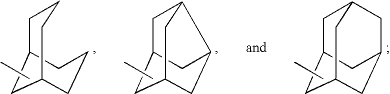

C₁₋₇alkoxy can be methoxy, ethoxy, propoxy, isopropoxy, butoxy, iso-butoxy, sec-butoxy, pentoxy, 3-pentoxy, hexyloxy, 1-methylhexyloxy, or heptyloxy; —C(═O)alkyl or (C₂₋₇)alkanoyl can be acetyl, propanoyl, butanoyl, pentanoyl, 4-methylpentanoyl, hexanoyl, or heptanoyl; aryl (Ar) can be phenyl, naphthyl, anthracenyl, phenanthrenyl, fluorenyl, tetrahydronaphthyl, or indanyl; Het can be pyrrolidinyl, piperidinyl, morpholinyl, thiomorpholinyl, or heteroaryl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

Specifically, —(CH₂)ₓ— can be a —(C₁₋₂₀alkylene)- and like radical when x is an integer from 1 to about 20, which can be methylenyl, ethylenyl, propylenyl, butylenyl, pentylenyl, 3-pentylenyl, hexylenyl, heptylenyl, octylenyl, nonylenyl, decylenyl, and like homologs.

Specifically, —(CH₂)ₓ— can be a —(C₁₋₆alkylene)- when x is an integer from 1 to about 6, which can be methylenyl, ethylenyl, propylenyl, butylenyl, pentylenyl, 3-pentylenyl, or hexylenyl.

Specifically, —(CH₂)ₓ— can be a —(C₁₋₄alkylene)- when x is an integer from 1 to about 4, which can be methylenyl, ethylenyl, propylenyl, or butylenyl.

Specifically, —(CR═CR)_{y}— can be a substituted or unsubstituted —(C₂ alkylene)- such as an ethylenyl, where each R can be, for example, independently H, (C₁-C₆)alkyl, Ar or Het, and y is 1.

Specifically, —(CR═CR)_{y}— can be a substituted —(C₂ alkylene)-, where each R can be, for example, independently H, (C₁-C₆)alkyl, Ar or Het, and y is 1, such as an ethylenyl of the formula —(C(Ar)═CH)— or 13 (CH═C(Ar))—.

Specifically, —(CR═CR)_{y}— can be an unsubstituted —(C₂ alkylene)- such as an ethylenyl, where each R can be, for example, H, and y is an integer from 1.

A specific value for Het includes a five-(5), six-(6), or seven-(7) membered saturated or unsaturated heterocycle, or heteroaromatic ring containing 1, 2, 3, or 4 heteroatoms, for example, non-peroxide oxy, thio, sulfinyl, sulfonyl, and nitrogen; as well as a radical of an ortho-fused bicyclic heterocycle of about eight to twelve ring atoms derived therefrom, particularly a benz-derivative or one derived by fusing a propylene, trimethylene, tetramethylene or another monocyclic Het diradical thereto.

A specific compound of the formula (I) can be, for example,

(CH₃O)₃Si—(CH₂)₃—(CH═CH)—C(═O)—H.

Another specific compound of the formula (I) can be, for example,

(CH₃O)₃Si—(CH₂)₁₀—(CH═CH)₂—C(═O)—H.

Another specific compound of the formula (I) can be, for example,

(CH₃CH₂O)₃Si—(CH₂)₆—(CH═CAr)—C(═O)—H

or

(CH₃CH₂O)₃Si—(CH₂)₆—(CAr═CH)—C(═O)—H.

Another specific compound of the formula (I) can be, for example,

(CH₃CH₂O)₃Si—(CH₂)₆—(C(Het)=CH)—C(═O)—H

or

(CH₃CH₂O)₃Si—(CH₂)₆13 (CH═C(Het))-C(═O)—H.

A specific compound of the formula (II) can be, for example,

(CH₃O)₃Si—(CH₂)₁₀—(CH═CH)—C(—O—CH₂—)₂H.

Another specific compound of the formula (II) can be, for example,

(CH₃O)₃Si—(CH₂)₁₀—(CH═CH)—C(—O—CH₂—CH₃)₂H.

Another specific compound of the formula (II) can be, for example,

(CH₃O)₃Si—(CH₂)₃—(CH═CH)—C(—O—CH₂—CH₃)₂H.

Another specific compound of the formula (II) can be, for example,

(CH₃O)₃Si—C₆H₅—(CH₂)—(CH═CH)—C(—O—CH₃)₂H.

Another specific compound of the formula (II) can be, for example,

(CH₃O)₃Si—(CH₂)₃—C₆H₅—(CH═CH)—C(—O—CH₃)₂H.

The abovementioned specific compounds of the formula (I) or (II), and like compounds of the disclosure, can include a salt or salts thereof.

“Hydrocarbon,” “hydrocarbyl” and like terms, in the context of the unsaturated aldehyde compounds and modified surfaces of the disclosure, refer to unsaturated divalent moieties —R— in the general formula —R—C(═O)H, and can include, for example, saturated alkyl hydrocarbons, unsaturated alkyl hydrocarbons, aromatic or aryl hydrocarbons, alkyl substituted aryl hydrocarbons, alkoxy substituted aryl hydrocarbons, heteroalkyl hydrocarbons, heteroaromatic or heteroaryl hydrocarbons, alky substituted heteroaryl hydrocarbons, alkoxy substituted heteroaryl hydrocarbons, and like hydrocarbon moieties, or combinations thereof, and as illustrated herein. In embodiments, the hydrocarbon of the unsaturated aldehyde compounds and modified substrate surfaces thereof can be selected if desired to be the same, similar to, or at least chemically or physically compatible with those hydrocarbons, if any, contained in the substrate, such as an organic polymer such as an insulating, semiconducting, or conducting polymer or copolymer, an inorganic polymer such as a glass, an organic-inorganic hybrid polymer such as a organo substituted polysiloxane, or combinations thereof. Additionally or alternatively, the hydrocarbon and like substituents of the unsaturated aldehyde compound can be selected if desired to be the same, similar to, or at least chemically or physically compatible with the protein or like material targeted for immobilization on the modified surface of the article.

Compounds of the disclosure, such as the abovementioned compounds or their precursor compounds of formula (I) or (II), can be prepared as described and illustrated herein, for example in the scheme below, by procedures analogous thereto, or by many different procedures, including partial or related procedures in the mentioned publications or patents. All of the variables used in the scheme(s) are as defined below or elsewhere herein.

The divalent hydrocarbon unit —(Ar′)_{w}—(CH₂)ₓ—(CR═CR)_{y}— as part of the unsaturated aldehyde modified surface as illustrated herein can generally provide the resulting modified surface with distinctive sites, or the like surface structures, having high surface area and high biomolecule immobilization activity.

Other conditions suitable for formation and modification of the compounds, oligomers, copolymers, or like products of the disclosure, from a variety of starting materials or intermediates, as illustrated herein are known. For example, see Feiser and Feiser, “Reagents for Organic Synthesis”, Vol. 1, et seq., 1967; March, J. “Advanced Organic Chemistry,” John Wiley & Sons, 4^{th} ed. 1992; House, H. O., “Modem Synthetic Reactions,” 2^{nd} ed., W. A. Benjamin, New York, 1972; and Larock, R. C., “Comprehensive Organic Transformations,” 2^{nd} ed., 1999, Wiley-VCH Publishers, New York.

The starting materials employed in the synthetic methods described herein are commercially available, have been reported in the scientific literature, or can be prepared from readily available starting materials using procedures known in the field. It may be desirable to optionally use a protecting group during all or portions of the above described or alternative synthetic procedures. Such protecting groups and methods for their introduction and removal are well known in the art. See Greene, T. W.; Wutz, P. G. M. “Protecting Groups In Organic Synthesis,” 2^{nd} ed., 1991, New York, John Wiley & Sons, Inc.

The unsaturated aldehyde compounds or protected variants thereof, and their surface bound version, and articles of the present disclosure can be useful in other applications, for example, an organosilicone coating, a conversion coating, a passivating coating, a conditioning coating as used for example in gas or liquid chromatography, a coupling agent (e.g., see Pludemann, Silane Coupling Agents, (1982)), a surface modifier, a silicone elastomer or like rubber applications, such as articles or devices, and like applications. Unsaturated aldehydes are known in natural or synthetic products with various applications, including for example, medicinal (e.g., drugs), agricultural (e.g., mosquito repellent), colorants (carotenoids), immobilization chemistry (linking agent), and like applications.

The disclosure provides methods to conveniently synthesize α,β-unsaturated aldehyde bearing surfaces. Known methods for preparing unsaturated aldehydes include, for example, an aldol condensation (refs. 2-3), condensation of hydroxysilanes with olefins (ref. 4), vinylsilanes with carbonyl derivatives (ref. 5), and propargyl alcohol oxidation (ref. 6).

Scheme 1 shows an example of an α,β-unsaturated aldehyde synthesis as described in U.S. Pat. No. 4,369,226, which mentions an aldol condensation where the polyglutaraldehyde (PGL) product was used as a linker between protein and the surface (ref. 2). In the course of PGL synthesis in basic medium (pH 9-12), unsaturated aldehydes groups were formed in the polymer backbone. However, problems were encountered with this approach. First, there was a solubility issue where even modest concentrations of the polymer precipitated from solution. When harsher conditions were used to either increase soluble polymer concentrations or to push the reaction to completion two secondary reaction pathways and their corresponding products were noted. The first pathway was a retroaldol reaction, that is, retro-polymerization and thus lost of bulk properties. The second pathway was a Cannizaro reaction where carboxylate and alcohol moieties formed on the polymer backbone. So the surface becomes pH sensitive and can induce ionic interactions with biomolecules having, for example, non-specific responses when analyzing covalent fixation. Therefore, even if this substrate were commonly used practical limitations remain.

Silane compounds having an unsaturated aldehyde that can be formed in solution are mentioned in WO 2066/014367. This process is based on reaction of hydroxysilanes with olefins. The process requires a precise control of temperature, time of the reaction, and reagent addition rate. The products can be isolated by fractional distillation from crude. These and other constraints limit their general utility in preparing modified surfaces. Similar complications are noted for vinylsilane condensations with carbonyl derivatives, and for propargyl alcohol oxidation processes. Additionally, a metal co-reagent must be used and requires a high grade purity. Such requirements make the synthesis expensive, tedious, and impractical.

In embodiments, the disclosure provides methods for preparing surfaces having α,β-unsaturated aldehyde functionality comprising, for example, reacting a surface bound or associated saturated aldehyde with a phosphonium ylide, or like salts, to furnish an olefin and an inert by-product phosphine oxide. In this Wittig reaction (ref. 7) approach an ylide having a protected or unprotected aldehyde carbonyl group was reacted with a surface aldehyde. Thus an α,β-unsaturated aldehyde (protected or unprotected) can be prepared in a one-pot process which includes reaction of a phosphonium ylide (commercial or prepared in situ) with an aldehyde followed by acetal hydrolysis to produce the α,β-unsaturated aldehyde if required (Scheme 2). The process of the disclosure is of practical value in that, for example, the reactions conditions only require a controlled atmosphere. The reaction solvent selection can depend upon the surface, for example, to provide sufficient wetting to react with the ylide solution. The reaction takes place smoothly at room temperature and thus can avoid heating equipment or a heating step.

In embodiments, the disclosed process is applicable to biomolecular immobilization, for example, proteins, and protein structures, such as an antibody, an antigen, a receptor, a ligand, and like substances. The disclosed process provides a modified surface having an α,β-unsaturated aldehyde covalently attached thereto. The attached α,β-unsaturated aldehyde is considerably more reactive than the corresponding saturated aldehyde as discussed elsewhere in the disclosure. In embodiments, the disclosed α,β-unsaturated aldehyde compound and modified surfaces thereof can be further tailored by for example, changing the chemical structure of the hydrocarbyl backbone to render the modified surface selectivity more or less receptive to specific immobilization targets.

When grafting a protein on a surface is desired, for example, covalent immobilization of a biomolecule, one or more of four major methods have be used. For example, epoxide opening, thiol-maleimide adduct formation, amide bond formation with activated ester, and imine formation between aldehyde and amine (ref. 1). The first two methods are the easiest to apply but suffer in that the epoxide opening is typically not efficient at room temperature (i.e., longer incubation time) and in water phase (i.e., hydrolysis competition), and secondly the thiol-maleimide adduct requires the presence of free thiol on a protein, in most instances a cysteine-dithiol reduction, that is a coreagent is required. The activated ester approach requires preliminary activation of carboxylic acid with a coreagent, and has longer reaction times.

In the imine approach, i.e., Schiff base formation, the imine is pH sensitive and is in equilibrium in water with the unsaturated aldehyde precursor. Practically speaking, this process must be accomplished at a pH that permits Schiff base formation and one more step to reduce this imine. In the process of the present disclosure this inconvenience can be overcome by first creating a Schiff base, which adduct rearranges to form a covalently bonded 1,4 adduct.

Scheme 1 shows a polyglutaraldehyde (PGL) synthesis under basic conditions. Secondary reactions have been reported and include a retro-aldol depolymerization (upper), and a Cannizaro reaction (lower) which furnishes aldehyde products additionally having allylic alcohol and carboxylic groups.

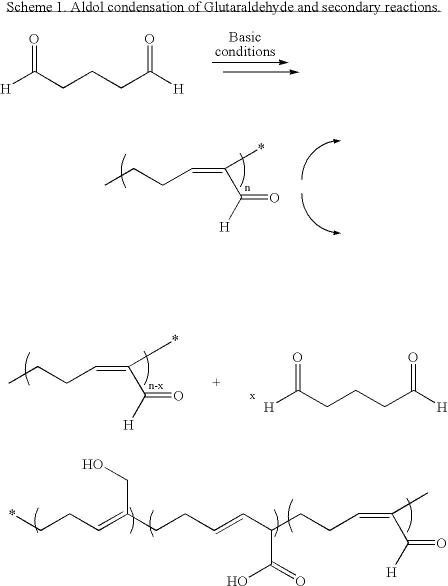

Scheme 2 shows a Wittig reaction used to prepare an unsaturated aldehyde. An ylide prepared from a phosphonium salt in presence of a strong base, reacts with an aldehyde to produce an olefin product having an aldehyde (R=—C(═O)H)) or masked aldehyde (R=ketal —C(—O—CH₂)₂H)) substituent.

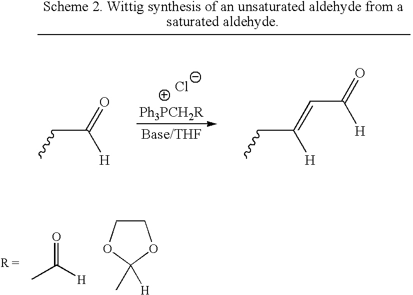

Scheme 3 compares mechanisms of saturated and unsaturated aldehyde protein immobilization. With saturated aldehydes (upper), an equilibrium exists between the aldehyde and the imine (aldehyde-protein) adduct. Thus a coreagent, such as a reducing agent may be necessary to drive the equilibrium toward a product with a stable bond, i.e., an amine protein adduct. With unsaturated aldehydes (lower), a second protein molecule (HX-Protein) adds to the imine adduct in 1,4- or conjugate addition fashion. The mechanism affords a stable 1,4-amine protein adduct of the now saturated aldehyde, which product does not require a separate reducing reagent to drive an unfavorable equilibrium.

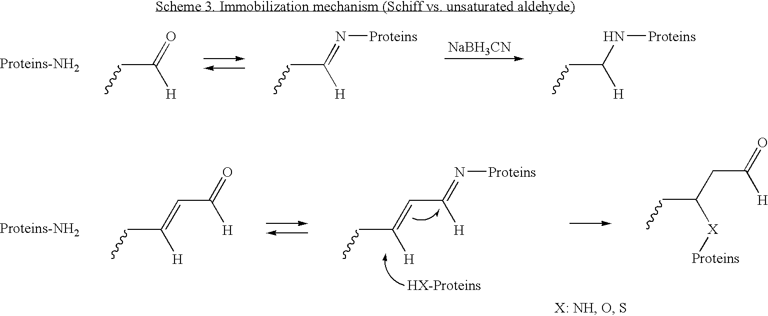

Scheme 4. An epoxysilane is first attached to a glass-slide. Next the epoxy function is converted into a saturated aldehyde. In presence of phosphonium salt and base, a protected unsaturated aldehyde is obtained. The protected unsaturated aldehyde can be readily deprotected with acid to afford the unsaturated aldehyde bearing surface.

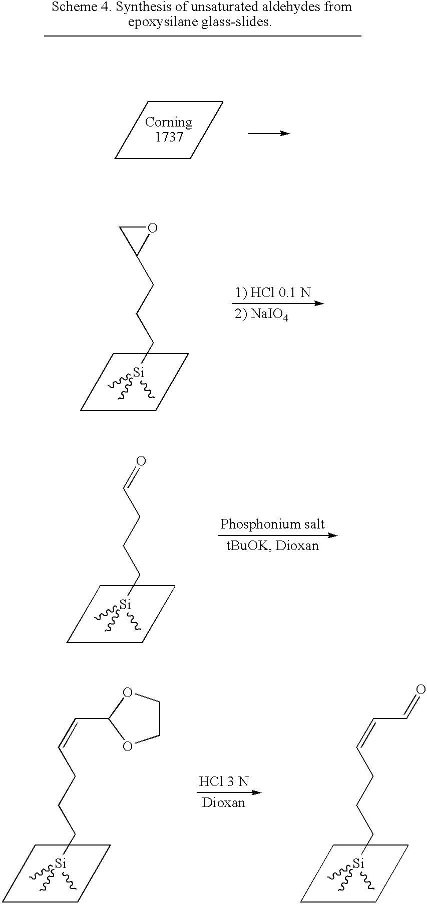

For additional definitions, descriptions, and methods of silica materials and related metal oxide materials, see for example, R. K. Iler, The Chemistry of Silica, Wiley-Interscience, 1979.

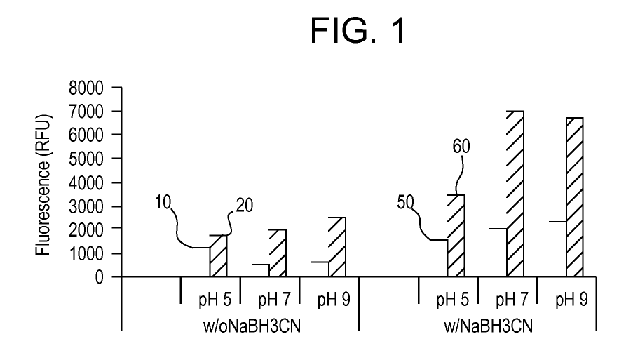

FIG. 1 shows comparative and exemplary protein immobilization results for modified surfaces, specifically, Cy5-SA immobilization results on a comparative saturated-aldehyde modified surface compared to an exemplary unsaturated-aldehyde modified surface. The unsaturated aldehyde surface had from about a 3 to about 4 fold greater capacity to immobilize protein compared to the saturated aldehyde surface.

When the saturated aldehyde surface or the unsaturated aldehyde surface was used for protein immobilization in the presence of a reducing agent, such as NaBH₃CN, increased fluorescence was observed following immobilization, which indicated that incremental and substantial, respectively, increases in immobilization capacity was obtained.

One method to immobilize proteins on a surface is the condensation of a surface aldehyde and an amine from a protein. Such a condensation allows one to attach biomolecules onto a surface via a Schiff base (imine). However, the imine can be labile in water (equilibrium) and can compromise proteins immobilization efficiency and stability. To solve this problem the disclosure provides a process to convert a saturated aldehyde into an α,βunsaturated aldehyde. The process of immobilizing protein with an α,β-unsaturated aldehyde modified surface creates a stable covalent bond between the immobilized biomolecule and the β-carbon of the α,β-unsaturated aldehyde of the supporting substrate.

### EXAMPLES

The following examples serve to more fully describe the manner of using the above-described disclosure, as well as to set forth the best modes contemplated for carrying out various aspects of the disclosure. It is understood that these examples in no way serve to limit the true scope of this disclosure, but rather are presented for illustrative purposes.

The following example illustrates one embodiment of the process for preparing saturated aldehyde groups on a substrate surface, such as a glass slide surface, and the process for converting the saturated aldehyde groups into unsaturated aldehydes (see Scheme 4).

Aldehyde Substrate Formation In a cuple jar was placed 1 mL of, 3-Glycidoxypropyltrimethoxysilane and 5 slides of pyrolyzed Corning® glass code 1737 and subjected to vapor deposition for 3 hour, at 100° C. After thorough washing with ethanol, the glass slides were dried with compressed air to afford an epoxysilane modified glass surface.

The epoxysilane surface was converted to the diol derivative by immersing the 5 epoxysilane modified glass-slides in a solution of HCl 0.1 N, at 80° C. for 2 hour. The glass slide substrates were washed with water and air-dried.

Finally, the diol substrates were converted to saturated aldehyde substrates by immersion of the 5 glass-slides in a water solution of NaIO₄ oxidant (1.5 g in 30 mL of deionized water). After 1 hour at about 25° C., the slides were washed with deionized water and air-dried.

### General Procedure for Saturated Aldehyde to Unsaturated Aldehyde Conversion

A saturated aldehyde substrate can be modified to an unsaturated aldehyde of the disclosure as follow. An aldehyde substrate, such as prepared above or as provided commercially, is dipped into an organic solvent (e.g., THF, dioxane) under inert atmosphere. A phosphonium salt (unprotected or protected aldehyde) is suspended in the solvent and then a strong base, such as t-BuOK or nBuLi, is added drop wise at 0° C. to form the phosphono ylide. The resulting mixture is stirred at room temperature for about 3 to 6 hours. Next the slides are washed with deionized water. Deprotection of the protected aldehyde can be accomplished with acid conditions (HCl 3N/aq/THF) for 3 hours, at room temperature, and followed by surface washing with deionized water.

### Example 1

Conversion of a Surface Bound Saturated Aldehyde to Unsaturated Aldehyde In a vessel, under inert atmosphere, three aldehyde glass slides were immersed in 60 mL of dry THF. To this solution, 0.5 g of phosphonium salt ((1,3-dioxolan-2-ylmethyl)triphenyl-phosphonium bromide) (1.18 mmol) was added. After dissolution, 600 microliters of tBuOK (20% wt in THF) (1.77 mmol) were added drop wise and the solution turned yellow. After 4 hours at RT, the slides were washed successively with water-ethanol-water and air-dried.

Final deprotection was done with a solution of HCl 3N in water, for 4 hours at room temperature. After washing with deionized water, the surfaces were air-dried. The supports are ready to use for biomolecular immobilization.

### Example 2

Modified Substrate Surface Wetting Properties The water wetting properties of a saturated and unsaturated aldehyde were compared. There is no apparent significant wetting difference (surface energy) between the saturated (native) and the unsaturated (final) aldehyde modified surfaces.

Table 1 shows that the chemical conversion from the original or native aldehyde modified glass-slide surface to an unsaturated aldehyde modified glass-slide surface does not adversely modify the wetting/hydrophobic properties.

TABLE 1
Water contact angle of aldehyde modified glass-slide surfaces

| Function | Native Aldehyde | Unsaturated Aldehyde |
|---|---|---|
| θw | 41-49 | 46-48 |

### Example 3

Immobilization Capacity Evaluation On the glass substrate, wells were delimited with a 12 wells silicon template (FlexiPerm). In each well, 75 microliters of an aqueous solution of Cytochrome 5-Streptavidin (Cy5-Sa) at 100 micrograms/mL was incubated for 2 hours. Evaluation was done with solutions at various pH values (5.5: acetate buffer; 7.4: PBS solution; 9.2: Borate buffer). After removing the buffer with suction, each well was washed with water and dried with compressed air. The slides were analyzed by fluorescence.

Immobilization Results Immobilization capacity of the modified surfaces were evaluated by measuring the fluorescence of Cy5-SA. Preliminary results showed protein immobilization with saturated aldehyde modified surfaces (10) having a level of 1,200 RFU (pH 5.5) and 500 (pH 7.4, pH 9.2) (FIG. 1). Protein immobilization data for the unsaturated aldehyde surface modified surfaces (20) having a level of 1,800 RFU (pH 5.5) and 2,000-2,500 RFU (pH 7.4, pH 9.2). The trend suggests about a 3 to 4 fold higher capacity for the unsaturated aldehyde surface compared to the saturated aldehyde surface.

Another comparison used a reducing agent. In presence of NaBH₃CN, the saturated aldehyde modified surfaces (50) showed an increased immobilization (1,600-2,100 RFU vs. 1,500-500 RFU, respectively) compared to immobilization on the saturated aldehyde surface modified surfaces (10) without reducing agent. However, this increased immobilization level was still lower compared to immobilization performed on unsaturated aldehyde modified surfaces (20) without a reducing agent (1,500-500 RFU vs. 1,800-2,500 RFU, respectively) present.

In one possible non-limiting mechanism (see Scheme 3), an equilibrium is present for both the saturated and the unsaturated aldehyde modified surfaces. To establish the full immobilization capacity of the unsaturated aldehyde modified surfaces, protein immobilization was also performed in presence of a reducing agent. The observed trend was about a 2 to about a 3 fold higher protein immobilization level with the unsaturated aldehyde modified surfaces (60) (1,700-2,500 RFU vs. 3,500-7,000 RFU) compared to the saturated aldehyde modified surfaces (50) when accomplished in the presence of a reducing agent.

From this data, the unsaturated aldehyde modified surfaces appeared to be more efficient for protein immobilization compared to the saturated aldehyde modified surface, even in presence of reducing agents.

The disclosure has been described with reference to various specific embodiments and techniques. However, it should be understood that many variations and modifications are possible while remaining within the spirit and scope of the disclosure.

### REFERENCES

1) “Mechanism of Inactivation of Monoamine Oxidase by trans-2-phenylcyclopropylamine and the Structure of the Enzyme-Inactivator Adduct.” Silverman, R. B., The Journal of Biological Chemistry, 1983, 258 (24), 14766-14769.
2) U.S. Pat. No. 5,665,781, “Use of Unsaturated Aldehyde and Alkanol Derivatives for Their Mosquito Repellency Properties.” assigned to International Flavors & Fragrances Inc.
3) U.S. Pat. No. 7,045,641, “Process for Preparing Polyenedialdehyde Monoacetals.” assigned to BASF Aktiengesellschaft.
4) EP 0,087,786 “Agarose-polyaldehyde beads, process for their production and their use.” assigned to Yeda Research and Development Company, Ltd.
5) [Requested]
6) U.S. Pat. No. 4,369,226, “Polyglutaraldehyde Synthesis and Protein Bonding Substrates.” assigned to California Institute of Technology.
7) U.S. Pat. No. 6,586,636 B2, “Aldol condensation,” assigned to Imperial Chemical Industries, PLC.
8) WO 2066/014367, “Functionalized Silicon Compounds.” assigned to Honeywell International.
9) “Organorhodium complex on smectite clay: preparation, characterization, and catalytic activity for the hydroformylation of vinylsilanes.” Valli, V. L. K., Alper, H., Chem. Mater., 1995, 7, 359-362.
10) EP 0952139A1; U.S. Pat. No. 6,118,027, “Preparation of Unsaturated Aldehydes from Propargyl Alcohol and Conjugated Diolefins.” assigned to Givaudan-Roure (International) SA.
11) “Chemical modification of functionalized copolymers with phosphonium groups by phase transfer catalysed Wittig reactions.” Ilia, G.; Popa, A.; Iliescu, S.; Dehelean, G.; Macarie, L.; Plesu, N., Mol. Cryst. Liq. Cryst. 2004, 416, 175-182.
12) U.S. Pat. No. 6,472,565 B1 “Method for Producing Aldehydes” assigned to Celanese Chemicals Europe, Gmbh.
13) U.S. Pat. No. 6,153,769 “Manufacture of Polyene Aldehydes,” assigned to Roche Vitamins Inc.
14) “Bioconjugate Techniques,” Greg T. Hermanson, 1996, Academic Press, Elsevier.

What is claimed is:

1. An article comprising:
a substrate having a surface modified with an unsaturated aldehyde of the formula:
≡Si—(CH₂)ₓ—(CR═CR)_{y}—C(═O)H
where
the ≡Si valences are associated with the substrate,
R is independently H, a substituted or unsubstituted, saturated or unsaturated (C₁-C₆)alkyl, Ar, or Het,
x is from 1 to about 20, and
y is from 1 to about 3.

2. The article of claim 1 wherein the substrate comprises at least one of a glass, a plastic, a metal, a ceramer, a composite, or combinations thereof.

3. The article of claim 1 wherein the surface modified with an unsaturated aldehyde has a thickness on the substrate of from about 3 to about 50 nanometers.

4. The article of claim 1 wherein the unsaturated aldehyde is of the formula:
≡Si—(CH₂)ₓ—(CR═CR)_{y}—C(═O)H
where
the ≡Si valences are associated with the substrate,
R is independently H, a substituted or unsubstituted, saturated or unsaturated (C₁-C₆)alkyl, Ar, or Het,
x is from 1 to about 6, and
y is from 1 to about 3.

5. The article of claim 4 wherein the unsaturated aldehyde is of the formula:
≡Si—(CH₂)₃—CH═CH—C(═O)H

6. A composition comprising the reaction product of a compound of the formula (I) or formula (II):
(R¹O)₃Si—(CH₂)ₓ—(CR═CR)_{y}—C(═O)H (I)
(R¹O)₃Si—(CH₂)ₓ—(CR═CR)_{y}—R² (II)
where
R is independently H, a substituted or unsubstituted, saturated or unsaturated (C₁-C₆)alkyl, Ar, or Het,
R¹ is independently H or (C₁-C₆)alkyl,
R² is an acetal of the formula —C(—O—R)(—O—R)—H where the acetal R groups are independently (C₁-C₆)alkyl or a conjoined cyclic R group,
x is from 1 to about 10, and
y is from 1 to about 3; and
a substrate comprised of at least one of a glass, a plastic, a metal, a ceramer, a composite, or combinations thereof.

7. The composition of claim 6 wherein the compound of the formula (I) or formula (II) is of the formula:
(R¹O)₃Si—(CH₂)₃—CH═CH—C(=O)H
or
(R¹O)₃Si—(CH₂)₃—CH═CH—C(—O—CH₂)₂H

8. A method of making an article, the method comprising:
reacting a substrate having a surface bearing a saturated aldehyde of the formula:
≡Si—(CH₂)ₓ—C(═O)H
where the ≡Si valences are associated with the substrate, and x is from 1 to about 10, with a “═CR—C(═O)—H” synthon to afford the article comprising a substrate having a surface modified with an α,β-unsaturated aldehyde of the formula
≡Si—(CH₂)ₓ—(CR═CR)_{y}—C(═O)H
where
R is independently H, a substituted or unsubstituted, saturated or unsaturated (C₁-C₆)alkyl, Ar, or Het,
x is from 1 to about 10, and
y is from 1 to about 3.

9. The method of claim 8 wherein the “═CR—C(═O)—H” synthon comprises an activated ylide precursor of at least one of:
a phosphonium salt of the formula
Ar₃P⁽⁺⁾—CR₂—R²X⁽⁻⁾ or Y₃P⁽⁺⁾—CR₂—R²X⁽⁻⁾
where
R is H and the other R is H, a substituted or unsubstituted, saturated or unsaturated (C₁-C₆)alkyl, Ar, or Het,
R² is an acetal of the formula —C(—O—R)(—O—R)—H where the acetal R groups are independently (C₁-C₆)alkyl or a conjoined cyclic R group,
Ar is aryl,
X is a counter anion, and
each Y is independently a substituted or unsubstituted (C₁-C₆)alkyl, a substituted or unsubstituted oxygenated (C₁-C₆)alkyl, a substituted or unsubstituted C₁₋₇alkoxy, a protected substituted or unsubstituted C₂₋₇alkanoyl, or where two Y groups form a cyclic (C₃-C₆)alkyl group or oxygenated cyclic (C₂-C₆)alkyl group;
a phosphonate of the formula
(RO)₂P(═O)—CR═CH—NH—R³
where
R is H, a substituted or unsubstituted, saturated or unsaturated (C₁-C₆)alkyl, Ar, or Het, and
R³ is (C₁-C₆)alkyl, (C₁-C₆)cyloalkyl, or Ar, or combinations thereof.

10. The method of claim 8 wherein the substrate comprises at least one of a glass, a plastic, a metal, a ceramer, a composite, or combinations thereof.

11. The method of claim 8 wherein the substrate has functional groups thereon that are reactive with compound of the formula (I) or formula (II), the functional groups being selected from hydroxyl, amine, hydrazide, and mixtures thereof.

12. A method of making an article comprising:
reacting a substrate with a compound of the formula (I) or formula (II):
(R¹O)₃Si—(Ar′)_{w}—(CH₂)ₓ—(CR═CR)_{y}—C(═O)H (I)
(R¹O)₃Si—(Ar′)_{w}—(CH₂)ₓ—(CR═CR)_{y}—R² (II)
where
R is independently H, a substituted or unsubstituted, saturated or unsaturated (C₁-C₆)alkyl, Ar, or Het,
R¹ is independently H or (C₁-C₆)alkyl,
R² is an acetal of the formula —C(—O—R)(—O—R)—H where the acetal R groups are independently (C₁-C₆)alkyl or a single conjoined cyclic R group,
Ar′ is aryl or Het,
w is from 0 to about 2,
x is from 1 to about 10, and
y is from 1 to about 3; and
the substrate comprises at least one of a glass, a plastic, a metal, a ceramer, a composite, or combinations thereof.

13. The method of claim 12 wherein the substrate has functional groups thereon reactive with a compound of the formula (I) or formula (II), the functional groups being selected from hydroxyl (—OH), amine (—NH₂ or —NHR), thiol (—SH), hydrazide (—R⁴R⁵N—NH₂ where at least one of R⁴ or R⁵ is acyl including carbonyl, sulfonyl, and phosphonyl derivatives), hydrazine (—R⁴R⁵N—NH₂), and combinations thereof.

14. The method of claim 12 further comprising hydrolyzing the intermediate acetal product resulting from formula (II).

15. The method of claim 12 wherein
R is independently H, a substituted or unsubstituted, saturated or unsaturated (C₁-C₆)alkyl,
R¹ is (C₁-C₆)alkyl,
R² is an acetal of the formula —C(—O—R)(—O—R)—H where the acetal R groups are independently (C₁-C₆)alkyl or a single cyclic R group,
w is from 0 to about 2,
x is from 1 to about 10, and
y is 1; and
the substrate comprises at least one of a glass, a plastic, a metal, a ceramer, a composite, or combinations thereof.

16. A method of immobilizing biomolecules, the method comprising:
contacting the article of claim 1 with a sample containing a biomolecule; and optionally
rinsing and drying the contacted article.

17. The method of claim 16 wherein the sample containing a biomolecule comprises at least a protein.
